(19) United States
(12) Patent Application Publication
(10) Pub. No.: US 2026/0358249 A1
(43) Pub. Date: Nov. 12, 2026

(54) MATERIAL LIGHT TRANSMITTANCE DETECTION DEVICE

(71) Applicant: Quanzhou Normal University, Quanzhou (CN)

(72) Inventors: Lina CUI, Quanzhou (CN); Canyi HUANG, Quanzhou (CN); Pinghui WU, Quanzhou (CN)

(21) Appl. No.: 19/326,875

(22) Filed: Sep. 12, 2025

(30) Foreign Application Priority Data
Sep. 14, 2024 (CN) 202411288025.6

(51) Int. Cl. G01N 21/59; G01N 21/15; G01N 33/36

(52) U.S. Cl. CPC G01N 21/59; G01N 21/15; G01N 33/367; G01N 2021/151; G01N 2021/152; G01N 2201/068

(57) ABSTRACT

A material light transmittance detection device comprises a light source cylinder and a C-shaped light shielding plate, a light source emitting seat is slidably arranged inside the light source cylinder, a light source receiving seat is slidably arranged on the C-shaped light shielding plate, a borosilicate glass ring is rotatably arranged between the light source cylinder and the C-shaped light shielding plate, and a fabric is arranged between the borosilicate glass ring and the C-shaped shading plate. The movement of the light source emitting seat drives the light source receiving seat to move, a light source emitted by a light source emitter is received by a light source receiver, and a detection range of the fabric can be changed along with the movement of the light source emitting seat, so that a detection range and a detection flexibility are greatly improved.

### CROSS REFERENCE TO RELATED APPLICATIONS

This application claims foreign priority of Chinese Patent Application No. 202411288025.6, filed on Sep. 14, 2024 in the China National Intellectual Property Administration, the disclosures of all of which are hereby incorporated by reference.

### TECHNICAL FIELD

The present invention relates to a detection device, is particularly a material light transmittance detection device, and belongs to the technical field of light transmittance detection.

### BACKGROUND OF THE PRESENT INVENTION

In a process of fabric detection, according to traditional methods, a produced fabric is cut and then detected by a detection device, or the machine is stopped to cut the fabric, which will waste a lot of time, thereby greatly reducing the detection efficiency. Moreover, in the process of detection, because the cutting of the fabric will produce more thread residues, once the thread residues are adsorbed on a detector, which affects a light transmittance, thereby affecting the detection quality. When the detector is cleaned, the detection efficiency will be further reduced because of wasting time.

Therefore, it is urgent to improve a material light transmittance detection device to solve the above problems.

### SUMMARY OF THE PRESENT INVENTION

The present invention aims to provide a material light transmittance detection device, a movement of a light source emitting seat drives a light source receiving seat to move, a light source emitted by a light source emitter is received by a light source receiver, and a detection range of a fabric can be changed along with the movement of the light source emitting seat, so that a detection range and a detection flexibility are greatly improved, and the fabric does not need to be cut, transferred, detected and the like, and meanwhile, inner and outer side surfaces of a borosilicate glass ring are dedusted, so that the detection efficiency and the detection accuracy are greatly improved.

In order to achieve the above objective, the main technical solution used in the present invention comprises:
a material light transmittance detection device, which comprises a light source cylinder and a C-shaped light shielding plate, wherein a light source emitting seat is slidably arranged inside the light source cylinder, a light source receiving seat corresponding to the light source cylinder is slidably arranged on the C-shaped light shielding plate, a borosilicate glass ring is rotatably arranged between the light source cylinder and the C-shaped light shielding plate, and a fabric is arranged between the borosilicate glass ring and the C-shaped shading plate;
the light source emitting seat is provided with an emitter hole, a light source emitter is fixedly arranged inside the emitter hole, the light source receiving seat is provided with a receiver hole corresponding to the emitter hole, a light source receiver is fixedly arranged inside the receiver hole, one side of the light source emitter is fixedly provided with first rubidium magnets symmetrically distributed, and one side of the light source receiver is fixedly provided with second rubidium magnets corresponding to the first rubidium magnets; and
a first guide plate is arranged inside the light source cylinder, the first guide plate is fixedly provided with a driving bar, the driving bar is fixedly provided with driving teeth, a driving motor groove is arranged inside the light source emitting seat, a driving motor is fixedly arranged inside the driving motor groove, an output end of the driving motor is provided with a driving gear, and the driving gear is meshed with the driving teeth.

Preferably, an outer side surface of the light source cylinder is provided with a glass groove, the borosilicate glass ring is rotatably arranged inside the glass groove, one side surface of the light source cylinder is provided with a light source cylinder light-transmitting hole, and the light source cylinder light-transmitting hole corresponds to the light source emitting seat; and
the C-shaped light shielding plate is provided with a light shielding plate light-transmitting hole, and the light shielding plate light-transmitting hole corresponds to the light source cylinder light-transmitting hole.

Preferably, a bottom portion of the light source emitting seat is provided with a first guide groove, the first guide plate is slidably arranged inside the first guide groove, one side of the light source emitting seat is provided with a second guide groove, a second guide plate is slidably arranged inside the second guide groove, and the second guide plate and the first guide plate are both fixedly arranged inside the light source cylinder.

Preferably, one side of the light source receiving seat is fixedly provided with a receiving seat connecting block, one side of the receiving seat connecting block far away from the light source receiving seat is fixedly provided with a signal processor, and the signal processor is electrically connected with the light source receiver;
the receiving seat connecting block is provided with receiving seat guide holes symmetrically distributed, a receiving seat guide rod is arranged inside the receiving seat guide hole, and the light source receiving seat is slidably arranged on the receiving seat guide rod; and
an outer side surface of the C-shaped light shielding plate is fixedly provided with a guide rod fixing seat, and the receiving seat guide rod is fixedly arranged on the guide rod fixing seat.

Preferably, a bottom portion of the light source cylinder is provided with a dust suction hole, and an interior of the light source cylinder is communicated with an inner side surface of the borosilicate glass ring through the dust suction hole; and
the dust suction hole is fixedly provided with a dedusting cylinder, the dedusting cylinder is provided with a dust suction groove, an interior of the dedusting cylinder is communicated with the dust suction hole through the dust suction groove, and one end of the interior of the dedusting cylinder is fixedly provided with an exhaust fan.

Preferably, one end of the dedusting cylinder is connected with a filter cover through a screw thread, an outer side surface of the filter cover is provided with filter cover through holes symmetrically distributed, the filter cover through hole is communicated with the interior of the dedusting cylinder, and a metal grid is fixedly arranged inside of the filter cover through hole; and
a steel wire ball layer is arranged inside the filter cover.

Preferably, one side surface of the light source cylinder is rotatably provided with a dust cleaning assembly, the dust cleaning assembly comprises a dry cotton cloth roll, an electrostatic roll, a sponge roll and a wet cotton cloth roll, and the dry cotton cloth roll, the electrostatic roll, the sponge roll and the wet cotton cloth roll abut against an outer side surface of the borosilicate glass ring.

Preferably, two side surfaces of the light source cylinder are both fixedly provided with a supporting frame, and the supporting frame is fixedly arranged on the light source cylinder through a welding block;
an upper end of the supporting frame is connected with a receiving seat supporting arm, and the receiving seat supporting arm is fixedly connected with the guide rod fixing seat by welding; and
a bottom portion of the supporting frame is provided with a plurality of assembly holes uniformly distributed.

Preferably, an upper side surface of the C-shaped light shielding plate is provided with L-shaped light shielding plates symmetrically distributed, and the L-shaped light shielding plate does not contact with the receiving seat connecting block.

Preferably, an upper side surface of the bottom portion of the supporting frame is provided with a guide roll, the guide roll is rotatably arranged on the supporting frame through a guide roll supporting seat, and the guide roll abuts against the fabric.

The present invention at least has the following beneficial effects.
1. In a process of detection, the fabric is wound on the borosilicate glass ring, so as to be able to rotate together with the borosilicate glass ring; the borosilicate glass ring is made of a glass material with high heat resistance, thereby having a low thermal expansion coefficient and maintaining relatively stable physical properties at high temperature, so that the borosilicate glass ring can not only guide the movement of the fabric, but also has a certain high permeability, so as to improve a refractive index of light; and in a production process of the fabric, the fabric can be directly detected without cutting the fabric, so that the detection flexibility is improved.
2. In order to improve the detection range and the detection flexibility, the light source emitter and the light source receiver need to correspond to each other, the light source emitting seat is fixedly provided with the first rubidium magnet, the light source receiving seat is provided with the second rubidium magnet corresponding to the first rubidium magnet, and one sides of the first rubidium magnet and the second rubidium magnet close to each other are magnetically opposite, so that the rubidium magnets attract each other under an action of magnetic force; and the movement of the light source emitting seat can drive the light source receiving seat to move at the same time, so that the detection range and the detection flexibility can be greatly improved, and the movement of the light source receiving seat driven by the movement of the light source emitting seat in the process of detection avoids extra energy consumption and improves a utilization rate of resources.
3. The movement of the light source emitting seat inside the light source cylinder can drive the light source receiving seat to move, and after the movement of the light source emitting seat, the light source emitted by the light source emitter on the light source emitting seat is received by the light source receiver on the light source receiving seat, then a light transmittance of the fabric is detected, and a detection range of the fabric may be changed along with the movement of the light source emitting seat, so that the detection range and the detection flexibility are greatly improved; and the fabric does not need to be cut, transferred, detected and the like in the process of detection, and the fabric is detected when the fabric is rolled up, so that the detection efficiency is greatly improved.
4. The light source cylinder is provided with the glass groove, the borosilicate glass ring is rotatably arranged inside the glass groove, the fabric abuts against the borosilicate glass ring, and the borosilicate glass ring rotates along with the movement of the fabric, so that friction between the fabric and the borosilicate glass ring is reduced, thereby avoiding a surface of the borosilicate glass ring from being scratched and improving the detection accuracy.
5. The light source emitting seat is arranged inside the light source cylinder, and light rays can only be emitted out through the light source cylinder light-transmitting hole, so that a light shielding performance is improved; moreover, the C-shaped light shielding plate has a C-shaped structure, and the light rays can only be emitted out through the light shielding plate light-transmitting hole, so that a certain light shielding performance is also achieved, thereby greatly improving the detection efficiency; and two sides of the light shielding plate light-transmitting hole are both fixedly provided with the L-shaped light shielding plate, which can not only play a certain guiding role in the sliding of the light source receiving seat, but also play a certain light shielding role, so that the detection accuracy is improved.
6. The outer side surface of the borosilicate glass ring is rotatably provided with the dust cleaning assembly, and the wet cotton cloth roll wets the outer side surface of the borosilicate glass ring first to wet impurities such as the thread residues, so as to prevent too much thread residues, dust and the like from floating in the air; the sponge roll absorbs moisture of the outer side surface of the borosilicate glass ring to reduce a humidity of the outer side surface of the borosilicate glass ring, and the electrostatic roll performs electrostatic adsorption to prevent dust from accumulating on the outer side surface of the borosilicate glass ring, so that the detection accuracy is improved; and then, the dry cotton cloth roll wipes the outer side surface of the borosilicate glass ring to improve the detection accuracy.
7. After the dust enters the interior of the light source cylinder, the dust enters the interior of the dedusting cylinder through the dust suction groove, one end of the interior of the dedusting cylinder is fixedly provided with the exhaust fan, and after the exhaust fan is started, the dust can enter the interior of the dedusting cylinder through the dust suction hole and the dust suction groove, and then is discharged through the exhaust fan, so that the purpose of dedusting can be achieved.

### DESCRIPTION OF THE DRAWINGS

The drawings described herein are used for providing further understanding for the present application, and constitute a part of the present application. Exemplary embodiments of the present application and the descriptions thereof are used for explaining the present application, and do not constitute any inappropriate limitation to the present application. In the drawings:

FIG. 1 is a first partial section view of the present invention;

FIG. 2 is a second partial section view of the present invention;

FIG. 3 is a stereoscopic diagram of the present invention;

FIG. 4 is a structural diagram of a light source cylinder of the present invention;

FIG. 5 is a structural diagram of a first guide plate of the present invention;

FIG. 6 is a structural diagram of a supporting frame of the present invention;

FIG. 7 is a structural diagram of a C-shaped light shielding plate of the present invention;

FIG. 8 is a structural diagram of a light source receiving seat of the present invention;

FIG. 9 is a stereoscopic diagram of a light source emitting seat of the present invention; and

FIG. 10 is a stereoscopic diagram of a dedusting cylinder of the present invention.

In the drawings, 1 refers to light source cylinder; 101 refers to glass groove; 102 refers to light source cylinder light-transmitting hole; 103 refers to second guide plate; 104 refers to first guide plate; 105 refers to driving bar; 106 refers to driving teeth; 107 refers to dust suction hole; 2 refers to C-shaped light shielding plate; 201 refers to light shielding plate light-transmitting hole; 202 refers to L-shaped light shielding plate; 203 refers to guide rod fixing seat; 3 refers to light source emitting seat; 301 refers to emitter hole; 302 refers to first rubidium magnet; 303 refers to second guide groove; 304 refers to first guide groove; 305 refers to driving motor groove; 306 refers to driving motor; 307 refers to driving gear; 308 refers to light source emitter; 4 refers to light source receiving seat; 401 refers to receiver hole; 402 refers to second rubidium magnet; 403 refers to receiving seat connecting block; 404 refers to signal processor; 405 refers to receiving seat guide rod; 406 refers to light source receiver; 407 refers to receiving seat guide hole; 5 refers to dedusting cylinder; 501 refers to exhaust fan; 502 refers to dust suction groove; 6 refers to filter cover; 601 refers to filter cover through hole; 602 refers to metal grid; 603 refers to steel wire ball layer; 7 refers to dust cleaning assembly; 701 refers to dry cotton cloth roll; 702 refers to electrostatic roll; 703 refers to sponge roll; 704 refers to wet cotton cloth roll; 8 refers to guide roll; 801 refers to guide roll supporting seat; 9 refers to supporting frame; 901 refers to welding block; 902 refers to receiving seat supporting arm; 903 refers to assembly hole; 10 refers to borosilicate glass ring; and 11 refers to fabric.

### DETAILED DESCRIPTION OF PREFERRED EMBODIMENTS

Implementation modes of the present application will be described in detail hereinafter with reference to the drawings and embodiments, so that a process of implementing the application of technical means to solve technical problems and achieve technical effects in the present application can be fully understand and executed accordingly.

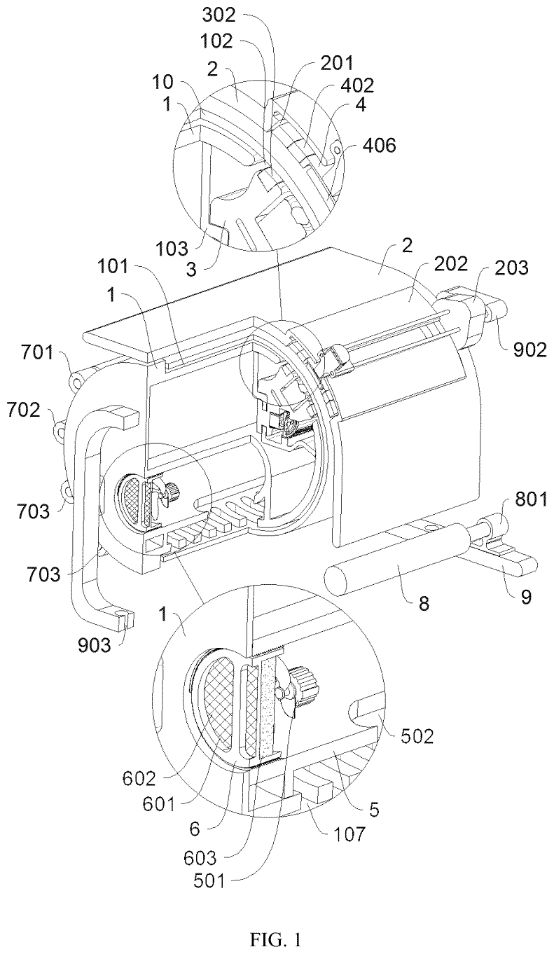

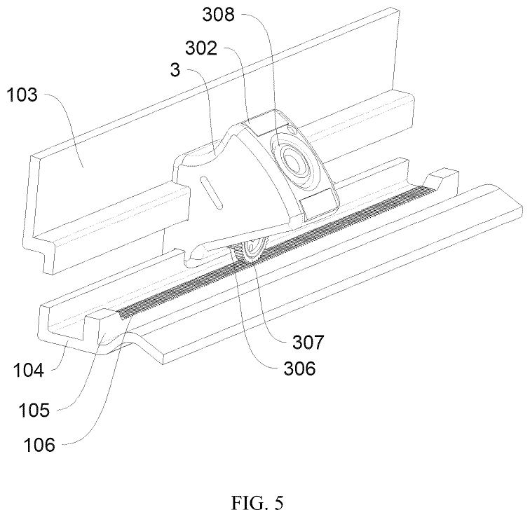

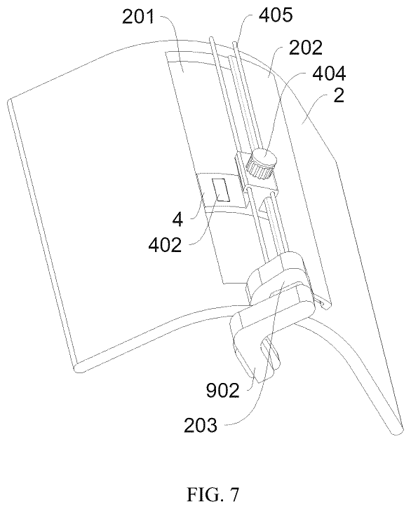

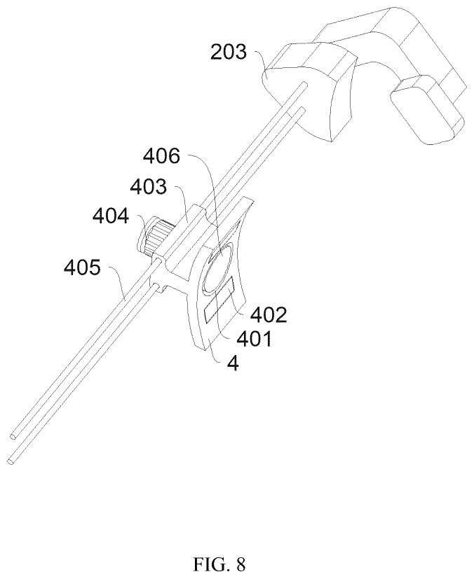

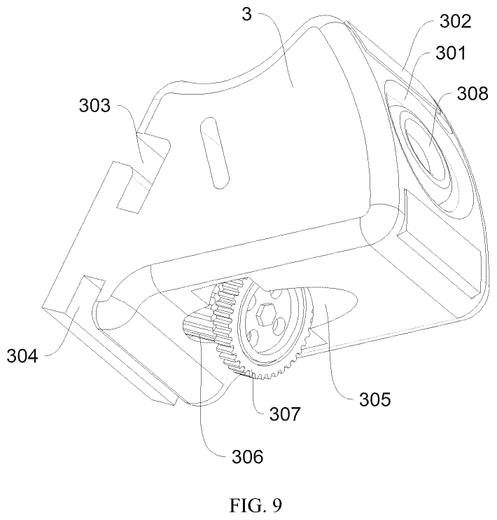

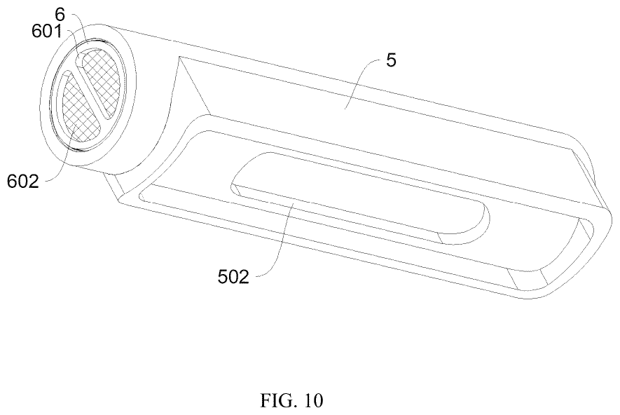

As shown in FIG. 1 to FIG. 10, a material light transmittance detection device provided by this embodiment comprises a light source cylinder 1 and a C-shaped light shielding plate 2, wherein a light source emitting seat 3 is slidably arranged inside the light source cylinder 1, and a light source receiving seat 4 corresponding to the light source cylinder 1 is slidably arranged on the C-shaped light shielding plate 2. During working, the light source emitting seat 3 emits light rays, and after the light rays pass through a to-be-tested material, transmission light and reflected light are received by the light source receiving seat 4, the light source receiving seat 4 converts a received light signal into an electrical signal, and a signal processor calculates a light transmittance according to the electrical signal. A borosilicate glass ring 10 is rotatably arranged between the light source cylinder 1 and the C-shaped light shielding plate 2, and a fabric 11 is arranged between the borosilicate glass ring 10 and the C-shaped shading plate 2. In a process of detection, the fabric 11 is wound on the borosilicate glass ring 10, so as to be able to rotate together with the borosilicate glass ring 10; the borosilicate glass ring 10 is made of a glass material with high heat resistance, thereby having a low thermal expansion coefficient and maintaining relatively stable physical properties at high temperature, so that the borosilicate glass ring can not only guide the movement of the fabric 11, but also has a certain high permeability, so as to improve a refractive index of light.

Meanwhile, in a production process of the fabric 11, the fabric 11 can be directly detected without cutting the fabric 11, so that the detection flexibility is improved.

The light source emitting seat 3 is provided with an emitter hole 301, a light source emitter 308 is fixedly arranged inside the emitter hole 301, the light source receiving seat 4 is provided with a receiver hole 401 corresponding to the emitter hole 301, and a light source receiver 406 is fixedly arranged inside the receiver hole 401. The light source emitter 308 is fixedly arranged inside the emitter hole 301 in the light source emitting seat 3, the light source receiver 406 is fixedly arranged inside the receiver hole 401 in the light source receiving seat 4, and the light source receiver 406 corresponds to the light source emitter 308. After the light source emitter 308 emits the light rays, the light rays are received by the light source receiver 406, that is to say, the light rays are emitted by the light source emitter 308, then pass through the borosilicate glass ring 10, and are received by the light source receiver 406, and the light transmittance of the fabric 11 is judged according to the light signal of the light rays received by the light source receiver 406. One side of the light source receiving seat 4 is fixedly provided with a receiving seat connecting block 403, one side surface of the receiving seat connecting block 403 far away from the light source receiving seat 4 is fixedly provided with a signal processor 404, and the signal processor 404 is electrically connected with the light source receiver 406. After the light source receiver 406 receives the signal, the signal is pushed to the signal processor 404, and the light signal is converted into the electrical signal. The signal processor 404 calculates the light transmittance according to the electrical signal, and results are displayed on a screen or output to an external device, so that the use convenience and the detection efficiency are improved.

In order to improve the detection range and the detection flexibility, the light source emitter 308 and the light source receiver 406 need to correspond to each other, one side of the light source emitter 308 is fixedly provided with first rubidium magnets 302 symmetrically distributed, and one side of the light source receiver 406 is fixedly provided with second rubidium magnets 402 corresponding to the first rubidium magnets 302. The light source emitting seat 3 is fixedly provided with the first rubidium magnet 302, the light source receiving seat 4 is provided with the second rubidium magnet 402 corresponding to the first rubidium magnet 302, and one sides of the first rubidium magnet 302 and the second rubidium magnet 402 close to each other are magnetically opposite, so that the rubidium magnets attract each other under an action of magnetic force. The movement of the light source emitting seat 3 can drive the light source receiving seat 4 to move at the same time, so that the detection range and the detection flexibility can be greatly improved.

In a process of driving the light source emitting seat 3, a first guide plate 104 is arranged inside the light source cylinder 1, the first guide plate 104 is fixedly provided with a driving bar 105, the driving bar 105 is fixedly provided with driving teeth 106, a driving motor groove 305 is arranged inside the light source emitting seat 3, a driving motor 306 is fixedly arranged inside the driving motor groove 305, an output end of the driving motor 306 is provided with a driving gear 307, and the driving gear 307 is meshed with the driving teeth 106. The light source emitting seat 3 is slidably arranged on the first guide plate 104, and the first guide plate 104 is fixedly arranged inside the light source cylinder 1, so that the movement stability of the light source emitting seat 3 is improved. The driving motor 306 is fixedly arranged inside the driving motor groove 305 of the light source emitting seat 3, and the driving gear 307 at the output end of the driving motor 306 is meshed with the driving teeth 106 on the driving bar 105, that is to say, when the driving motor 306 rotates, the driving teeth 106 on the driving bar 105 are meshed with the driving gear 307, so that the light source emitting seat 3 can move horizontally on the first guide plate 104, and the fabric 11 corresponding to the light source emitting seat 3 is detected after stopping the light source emitting seat 3, thereby greatly improving the detection range and the detection flexibility.

The movement of the light source receiving seat 4 driven by the movement of the light source emitting seat 3 in the process of detection avoids extra energy consumption and improves a utilization rate of resources. A process of detecting the fabric 11 is specifically as follows:
the driving gear 307 at the output end of the driving motor 306 is meshed with the driving teeth 106 on the driving bar 105, and after the driving motor groove 305 is started, the light source emitting seat 3 can move horizontally and circularly on the first guide plate 104, and the fabric 11 corresponding to the light source emitting seat 3 is detected after stopping the light source emitting seat 3.

A bottom portion of the light source emitting seat 3 is provided with a first guide groove 304, the first guide plate 104 is slidably arranged inside the first guide groove 304, one side of the light source emitting seat 3 is provided with a second guide groove 303, a second guide plate 103 is slidably arranged inside the second guide groove 303, and the second guide plate 103 and the first guide plate 104 are both fixedly arranged inside the light source cylinder 1. The second guide plate 103 and the first guide plate 104 are fixedly arranged inside the light source cylinder 1, and meanwhile, the second guide plate 103 is slidably arranged on the second guide groove 303, so that the movement stability of the light source emitting seat 3 is improved, thereby preventing the light source emitting seat 3 from being skewed during the movement.

The receiving seat connecting block 403 is provided with receiving seat guide holes 407 symmetrically distributed, a receiving seat guide rod 405 is arranged inside the receiving seat guide hole 407, and the light source receiving seat 4 is slidably arranged on the receiving seat guide rod 405. The receiving seat connecting block 403 is provided with the receiving seat guide hole 407 corresponding to the receiving seat guide rod 405, so that the receiving seat connecting block 403 can slide on the receiving seat guide rod 405, so that the stability of the light source receiving seat 4 is improved. An outer side surface of the C-shaped light shielding plate 2 is fixedly provided with a guide rod fixing seat 203, and the receiving seat guide rod 405 is fixedly arranged on the guide rod fixing seat 203. The receiving seat guide rod 405 is fixed on the guide rod fixing seat 203, so that a structural strength of the receiving seat guide rod 405 is improved, thereby preventing the light source receiving seat 4 from being skewed during the movement.

Therefore, the movement of the light source emitting seat 3 inside the light source cylinder 1 can drive the light source receiving seat 4 to move, and after the movement of the light source emitting seat 3, the light source emitted by the light source emitter 308 on the light source emitting seat 3 is received by the light source receiver 406 on the light source receiving seat 4, then a light transmittance of the fabric 11 is detected, and a detection range of the fabric 11 may be changed along with the movement of the light source emitting seat 3, so that the detection range and the detection flexibility are greatly improved; and the fabric 11 does not need to be cut, transferred, detected and the like in the process of detection, and the fabric is detected when the fabric 11 is rolled up, so that the detection efficiency is greatly improved.

In order to ensure the detection accuracy, an outer side surface of the light source cylinder 1 is provided with a glass groove 101, the borosilicate glass ring 10 is rotatably arranged inside the glass groove 101, one side surface of the light source cylinder 1 is provided with a light source cylinder light-transmitting hole 102, and the light source cylinder light-transmitting hole 102 corresponds to the light source emitting seat 3. The light source cylinder 1 is provided with the glass groove 101, the borosilicate glass ring 10 is rotatably arranged inside the glass groove 101, the fabric 11 abuts against the borosilicate glass ring 10, and the borosilicate glass ring 10 rotates along with the movement of the fabric 11, so that friction between the fabric 11 and the borosilicate glass ring 10 is reduced, thereby avoiding a surface of the borosilicate glass ring 10 from being scratched and improving the detection accuracy.

Meanwhile, the C-shaped light shielding plate 2 is provided with a light shielding plate light-transmitting hole 201, and the light shielding plate light-transmitting hole 201 corresponds to the light source cylinder light-transmitting hole 102. The light rays emitted by the light source emitter 308 on the light source emitting seat 3 pass through the light source cylinder light-transmitting hole 102, then pass through the borosilicate glass ring 10, pass through the light shielding plate light-transmitting hole 201, and are received by the light source receiver 406, so that the light rays pass through the light source cylinder light-transmitting hole 102 and the light shielding plate light-transmitting hole 201, thereby reducing a loss of the light rays.

In addition, the light source emitting seat 3 is arranged inside the light source cylinder 1, and the light rays can only be emitted out through the light source cylinder light-transmitting hole 102, so that a light shielding performance is improved; moreover, the C-shaped light shielding plate 2 has a C-shaped structure, and the light rays can only be emitted out through the light shielding plate light-transmitting hole 201, so that a certain light shielding performance is also achieved, thereby greatly improving the detection efficiency ; an upper side surface of the C-shaped light shielding plate 2 is provided with L-shaped light shielding plates 202 symmetrically distributed, and the L-shaped light shielding plate 202 does not contact with the receiving seat connecting block 403; and two sides of the light shielding plate light-transmitting hole 201 are both fixedly provided with the L-shaped light shielding plate 202, which can not only play a certain guiding role in the sliding of the light source receiving seat 4, but also play a certain light shielding role, so that the detection accuracy is improved.

Further, because the fabric 11 is wound on the borosilicate glass ring 10, thread residues and impurities on the fabric 11 may be attached to the surface of the borosilicate glass ring 10, and the increase of the impurities will affect the light transmittance, so that the detection accuracy is reduced. In order to reduce an error of the light transmittance, it is necessary to dedust the outer and inner side surfaces of the borosilicate glass ring 10.

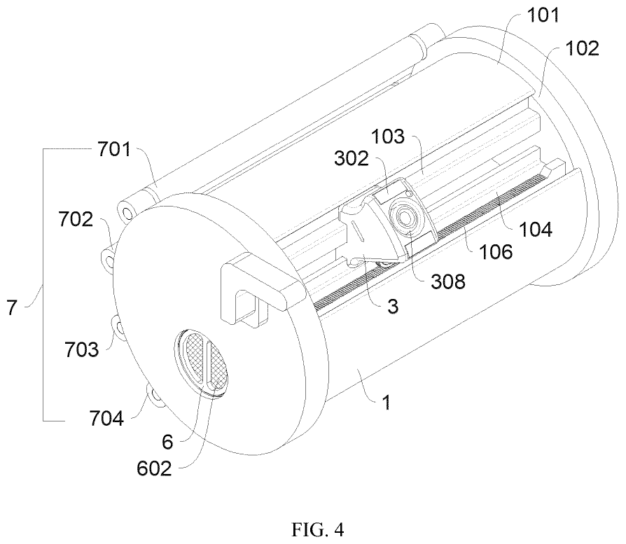

When the outer side surface of the borosilicate glass ring 10 is dedusted:
as shown in FIG. 4, one side surface of the light source cylinder 1 is rotatably provided with a dust cleaning assembly 7, the dust cleaning assembly 7 comprises a dry cotton cloth roll 701, an electrostatic roll 702, a sponge roll 703 and a wet cotton cloth roll 704, and the dry cotton cloth roll 701, the electrostatic roll 702, the sponge roll 703 and the wet cotton cloth roll 704 abut against an outer side surface of the borosilicate glass ring 10. The outer side surface of the borosilicate glass ring 10 is rotatably provided with the dust cleaning assembly 7, and an external dedusting process is specifically as follows:
wetting of impurities: the wet cotton cloth roll 704 on the dust cleaning assembly 7 wets the outer side surface of the borosilicate glass ring 10 first to wet the impurities such as the thread residues, so as to prevent too much thread residues, dust and the like from floating in the air to affect the detection accuracy;
cleaning of glass ring: the sponge roll 703 absorbs moisture of the outer side surface of the borosilicate glass ring 10, and cleans the wetted thread residues and impurities together, so that a humidity of the outer side surface of the borosilicate glass ring 10 is reduced at the same time;
electrostatic adsorption: the electrostatic roll 702 performs electrostatic adsorption on fine dust to prevent the dust from accumulating on the outer side surface of the borosilicate glass ring 10, so that the detection accuracy is improved; and
wiping of glass ring: after the outer side surface of the borosilicate glass ring 10 is dedusted by the electrostatic roll 702, the dry cotton cloth roll 701 wipes the outer side surface of the borosilicate glass ring 10 to improve the detection accuracy.

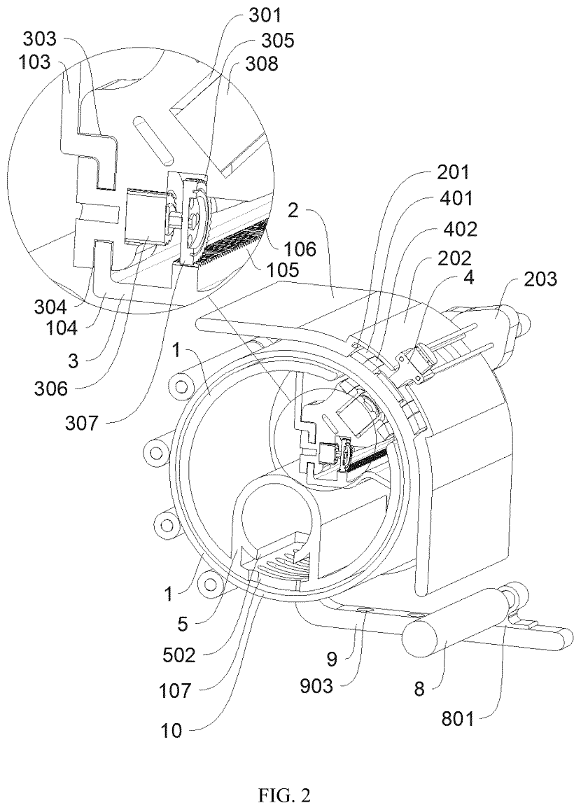

When the inner side surface of the borosilicate glass ring 10 is dedusted:
as shown in FIG. 1 and FIG. 2, in the process of detection, a small amount of thread residues may enter an interior of the light source cylinder 1, a bottom portion of the light source cylinder 1 is provided with a dust suction hole 107, and the interior of the light source cylinder 1 is communicated with the inner side surface of the borosilicate glass ring 10 through the dust suction hole 107. The light source cylinder 1 is communicated with the inner side surface of the borosilicate glass ring 10 through the dust suction hole 107, so that the dust on the inner side surface of the borosilicate glass ring 10 can enter the interior of the light source cylinder 1 through the dust suction hole 107. The dust suction hole 107 is fixedly provided with a dedusting cylinder 5, and the dedusting cylinder 5 is provided with a dust suction groove 502. After the dust enters the interior of the light source cylinder 1, the dust enters an interior of the dedusting cylinder 5 through the dust suction groove 502. The interior of the dedusting cylinder 5 is communicated with the dust suction hole 107 through the dust suction groove 502, and one end of the interior of the dedusting cylinder 5 is fixedly provided with an exhaust fan 501. One end of the interior of the dedusting cylinder 5 is fixedly provided with the exhaust fan 501, and after the exhaust fan 501 is started, the dust can enter the interior of the dedusting cylinder 5 through the dust suction hole 107 and the dust suction groove 502, and then is discharged through the exhaust fan 501, so that the purpose of dedusting can be achieved.

In addition, one end of the dedusting cylinder 5 is connected with a filter cover 6 through a screw thread, an outer side surface of the filter cover 6 is provided with filter cover through holes 601 symmetrically distributed, the filter cover through hole 601 is communicated with the interior of the dedusting cylinder 5, and a metal grid 602 is fixedly arranged inside of the filter cover through hole 601; and a steel wire ball layer 603 is arranged inside the filter cover 6. One end of the dedusting cylinder 5 is connected with the filter cover 6 through the screw thread, which facilitates disassembling the filter cover 6. The air inside the dedusting cylinder 5 can be exhausted through the filter cover through hole 601 in the filter cover 6, and the metal grid 602 on the filter cover through hole 601 plays a certain filtering role, thereby avoiding the dust from entering the air to attach to the side surface of the borosilicate glass ring 10 again. The steel wire ball inside the filter cover 6 may filter the air to block impurities such as dust in the air inside the steel wire ball layer 603, and then the steel wire ball layer 603 inside the filter cover 6 is replaced after the filter cover 6 is removed, which facilitates cleaning the steel wire ball layer 603 in the later stage, so that the dust on the inner side surface of the borosilicate glass ring 10 can be cleaned, thereby improving the detection accuracy.

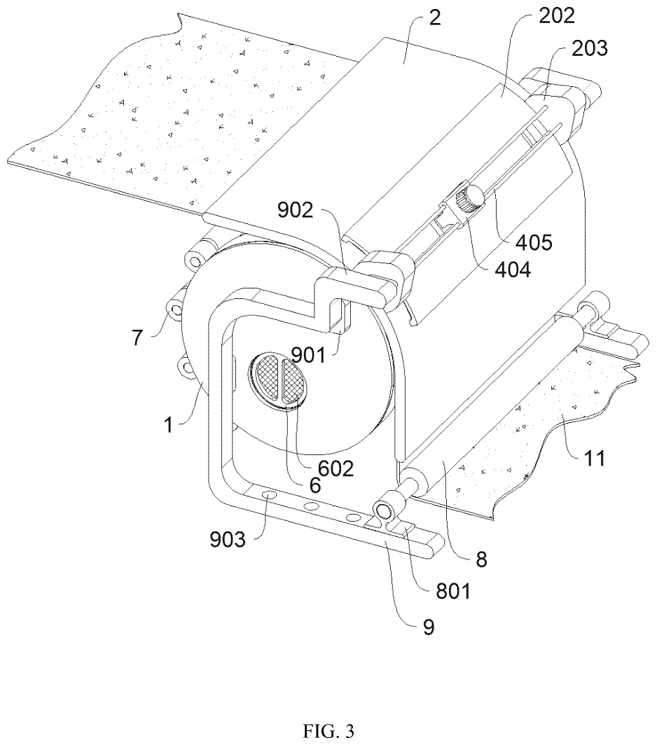

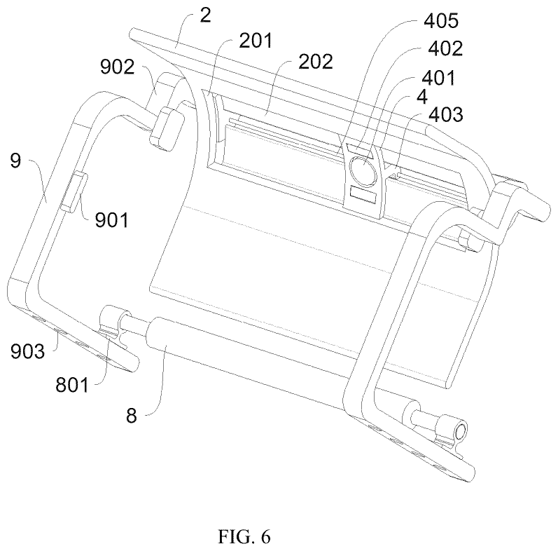

Further, as shown in FIG. 3 and FIG. 6, two side surfaces of the light source cylinder 1 are both fixedly provided with a supporting frame 9, and the supporting frame 9 is fixedly arranged on the light source cylinder 1 through a welding block 901. The two side surfaces of the light source cylinder 1 are fixed by the supporting frames 9, so that the stability of the light source cylinder 1 is improved.

Meanwhile, an upper end of the supporting frame 9 is connected with a receiving seat supporting arm 902, and the receiving seat supporting arm 902 is fixedly connected with the guide rod fixing seat 203 by welding. The guide rod fixing seat 203 is fixed on the C-shaped light shielding plate 2, and the guide rod fixing seat 203 is fixed on the supporting frame 9 at the same time. A bottom portion of the supporting frame 9 is provided with a plurality of assembly holes 903 uniformly distributed, and then the whole device is fixed in front of a winding roll of the fabric 11 through the assembly holes 903 in the supporting frame 9, so that the whole device can be directly fixed through the supporting frame 9, that is, the overall stability of the device can be improved, and a large amount of space can be saved and a utilization rate of space can be improved at the same time.

In addition, an upper side surface of the bottom portion of the supporting frame 9 is provided with a guide roll 8, the guide roll 8 is rotatably arranged on the supporting frame 9 through a guide roll supporting seat 801, and the guide roll 8 abuts against the fabric 11. The guide roll 8 on the supporting frame 9 guides the fabric 11 to a certain extent, and because one side surface of the fabric 11 abuts against the borosilicate glass ring 10, the guide roll 8 can prevent the fabric 11 from contacting with the C-shaped light shielding plate 2, which improves the detection accuracy on one hand and prevents the fabric from being damaged due to the friction between the fabric 11 and the C-shaped light shielding plate 2 on the other hand, and facilitates timely treatment of the fabric 11, thereby improving the use convenience.

As shown in FIG. 1 to FIG. 10, a principle of the material light transmittance detection device provided by this embodiment is as follows:
in a process of detection, the fabric 11 is wound on the borosilicate glass ring 10, so as to be able to rotate together with the borosilicate glass ring 10; the borosilicate glass ring 10 is made of a glass material with high heat resistance, thereby having a low thermal expansion coefficient and maintaining relatively stable physical properties at high temperature, so that the borosilicate glass ring can not only guide the movement of the fabric 11, but also has a certain high permeability, so as to improve a refractive index of light.

Meanwhile, in a production process of the fabric 11, the fabric 11 can be directly detected without cutting the fabric 11, so that the detection flexibility is improved.

The light source emitter 308 is fixedly arranged inside the emitter hole 301 in the light source emitting seat 3, the light source receiver 406 is fixedly arranged inside the receiver hole 401 in the light source receiving seat 4, and the light source receiver 406 corresponds to the light source emitter 308. After the light source emitter 308 emits the light rays, the light rays are received by the light source receiver 406, that is to say, the light rays are emitted by the light source emitter 308, then pass through the borosilicate glass ring 10, and are received by the light source receiver 406, and the light transmittance of the fabric 11 is judged according to the light signal of the light rays received by the light source receiver 406. After the light processor 404 receives the signal, the signal is pushed to the signal processor 404, and the light signal is converted into the electrical signal. The signal processor 404 calculates the light transmittance according to the electrical signal, and results are displayed on a screen or output to an external device, so that the use convenience and the detection efficiency are improved.

One sides of the first rubidium magnet 302 and the second rubidium magnet 402 close to each other are magnetically opposite, so that the rubidium magnets attract each other under an action of magnetic force; and the movement of the light source emitting seat 3 can drive the light source receiving seat 4 to move at the same time, so that the detection range and the detection flexibility can be greatly improved.

The movement of the light source emitting seat 3 inside the light source cylinder 1 can drive the light source receiving seat 4 to move, and after the movement of the light source emitting seat 3, the light source emitted by the light source emitter 308 on the light source emitting seat 3 is received by the light source receiver 406 on the light source receiving seat 4, then a light transmittance of the fabric 11 is detected, and a detection range of the fabric 11 may be changed along with the movement of the light source emitting seat 3, so that the detection range and the detection flexibility are greatly improved; and the fabric 11 does not need to be cut, transferred, detected and the like in the process of detection, and the fabric 11 is detected when the fabric 11 is rolled up, so that the detection efficiency is greatly improved.

If some terms are used to refer to specific components in the specification and the claims, those skilled in the art should understand that hardware manufacturers may refer to the same component by different terms. In the specification and the claims, the components are distinguished by the difference of functions of the components instead of the difference of names. For example, the term “comprising” mentioned throughout the specification and the claims is an open term, and should be interpreted as “comprising but being not limited to”. The term “about” refers to being within an acceptable error range, and those skilled in the art can solve the technical problems within the movement error range, thereby basically achieving the technical effects.

It should be further noted that the terms “include”, “comprise”, or any variants thereof are intended to cover a non-exclusive inclusion. Therefore, a commodity or a system that includes a series of elements not only includes such elements, but also includes other elements not specified expressly, or may include inherent elements of the commodity or the system. Unless otherwise specified, an element limited by “include a/an . . . ” does not exclude other same elements existing in the commodity or the system that includes the element.

The foregoing shows and describes several preferred embodiments of the present invention. However, as described above, it should be understood that the present invention is not limited to the forms disclosed herein, the preferred embodiments should not be regarded as excluding other embodiments, but can be used in various other combinations, modifications and environments, and can be changed by the above teachings or the technology or knowledge in related fields within the scope of concept of the present invention described herein. However, the modifications and changes made by those skilled in the art without departing from the spirit and scope of the present invention should fall within the scope of protection of the appended claims.

We claim:

1. A material light transmittance detection device, comprising a light source cylinder (1) and a C-shaped light shielding plate (2), wherein a light source emitting seat (3) is slidably arranged inside the light source cylinder (1), a light source receiving seat (4) corresponding to the light source cylinder (1) is slidably arranged on the C-shaped light shielding plate (2), a borosilicate glass ring (10) is rotatably arranged between the light source cylinder (1) and the C-shaped light shielding plate (2), and a fabric (11) is arranged between the borosilicate glass ring (10) and the C-shaped shading plate (2);
the light source emitting seat (3) is provided with an emitter hole (301), a light source emitter (308) is fixedly arranged inside the emitter hole (301), the light source receiving seat (4) is provided with a receiver hole (401) corresponding to the emitter hole (301), a light source receiver (406) is fixedly arranged inside the receiver hole (401), one side of the light source emitter (308) is fixedly provided with first rubidium magnets (302) symmetrically distributed, and one side of the light source receiver (406) is fixedly provided with second rubidium magnets (402) corresponding to the first rubidium magnets (302);
a first guide plate (104) is arranged inside the light source cylinder (1), the first guide plate (104) is fixedly provided with a driving bar (105), the driving bar (105) is fixedly provided with driving teeth (106), a driving motor groove (305) is arranged inside the light source emitting seat (3), a driving motor (306) is fixedly arranged inside the driving motor groove (305), an output end of the driving motor (306) is provided with a driving gear (307), and the driving gear (307) is meshed with the driving teeth (106);
an outer side surface of the light source cylinder (1) is provided with a glass groove (101), the borosilicate glass ring (10) is rotatably arranged inside the glass groove (101), one side surface of the light source cylinder (1) is provided with a light source cylinder light-transmitting hole (102), and the light source cylinder light-transmitting hole (102) corresponds to the light source emitting seat (3);
the C-shaped light shielding plate (2) is provided with a light shielding plate light-transmitting hole (201), and the light shielding plate light-transmitting hole (201) corresponds to the light source cylinder light-transmitting hole (102);
a bottom portion of the light source cylinder (1) is provided with a dust suction hole (107), and an interior of the light source cylinder (1) is communicated with an inner side surface of the borosilicate glass ring (10) through the dust suction hole (107); and
the dust suction hole (107) is fixedly provided with a dedusting cylinder (5), the dedusting cylinder (5) is provided with a dust suction groove (502), an interior of the dedusting cylinder (5) is communicated with the dust suction hole (107) through the dust suction groove (502), and one end of the interior of the dedusting cylinder (5) is fixedly provided with an exhaust fan (501).

2. The material light transmittance detection device according to claim 1, wherein a bottom portion of the light source emitting seat (3) is provided with a first guide groove (304), the first guide plate (104) is slidably arranged inside the first guide groove (304), one side of the light source emitting seat (3) is provided with a second guide groove (303), a second guide plate (103) is slidably arranged inside the second guide groove (303), and the second guide plate (103) and the first guide plate (104) are both fixedly arranged inside the light source cylinder (1).

3. The material light transmittance detection device according to claim 1, wherein one side of the light source receiving seat (4) is fixedly provided with a receiving seat connecting block (403), one side of the receiving seat connecting block (403) far away from the light source receiving seat (4) is fixedly provided with a signal processor (404), and the signal processor (404) is electrically connected with the light source receiver (406);
the receiving seat connecting block (403) is provided with receiving seat guide holes (407) symmetrically distributed, a receiving seat guide rod (405) is arranged inside the receiving seat guide hole (407), and the light source receiving seat (4) is slidably arranged on the receiving seat guide rod (405); and
an outer side surface of the C-shaped light shielding plate (2) is fixedly provided with a guide rod fixing seat (203), and the receiving seat guide rod (405) is fixedly arranged on the guide rod fixing seat (203).

4. The material light transmittance detection device according to claim 1, wherein one end of the dedusting cylinder (5) is connected with a filter cover (6) through a screw thread, an outer side surface of the filter cover (6) is provided with filter cover through holes (601) symmetrically distributed, the filter cover through hole (601) is communicated with the interior of the dedusting cylinder (5), and a metal grid (602) is fixedly arranged inside of the filter cover through hole (601); and
a steel wire ball layer (603) is arranged inside the filter cover (6).

5. The material light transmittance detection device according to claim 1, wherein one side surface of the light source cylinder (1) is rotatably provided with a dust cleaning assembly (7), the dust cleaning assembly (7) comprises a dry cotton cloth roll (701), an electrostatic roll (702), a sponge roll (703) and a wet cotton cloth roll (704), and the dry cotton cloth roll (701), the electrostatic roll (702), the sponge roll (703) and the wet cotton cloth roll (704) abut against an outer side surface of the borosilicate glass ring (10).

6. The material light transmittance detection device according to claim 3, wherein two side surfaces of the light source cylinder (1) are both fixedly provided with a supporting frame (9), and the supporting frame (9) is fixedly arranged on the light source cylinder (1) through a welding block (901);
an upper end of the supporting frame (9) is connected with a receiving seat supporting arm (902), and the receiving seat supporting arm (902) is fixedly connected with the guide rod fixing seat (203) by welding; and
a bottom portion of the supporting frame (9) is provided with a plurality of assembly holes (903) uniformly distributed.

7. The material light transmittance detection device according to claim 3, wherein an upper side surface of the C-shaped light shielding plate (2) is provided with L-shaped light shielding plates (202) symmetrically distributed, and the L-shaped light shielding plate (202) does not contact with the receiving seat connecting block (403).

8. The material light transmittance detection device according to claim 6, wherein an upper side surface of the bottom portion of the supporting frame (9) is provided with a guide roll (8), the guide roll (8) is rotatably arranged on the supporting frame (9) through a guide roll supporting seat (801), and the guide roll (8) abuts against the fabric (11).
